(19) United States
(12) Patent Application Publication
(10) Pub. No.: US 2025/0339259 A1
(43) Pub. Date: Nov. 6, 2025

(54) DEVICE FOR TISSUE SUPPORT

(71) Applicant: Buck Surgical, Inc., St. Louis, MO (US)

(72) Inventor: Donald W. Buck, St. Louis, MO (US)

(73) Assignee: Buck Surgical, Inc., St. Louis, MO (US)

(21) Appl. No.: 19/188,140

(22) Filed: Apr. 24, 2025

Related U.S. Application Data
(60) Provisional application No. 63/640,928, filed on May 1, 2024.

(51) Int. Cl. A61F 2/08

(52) U.S. Cl. CPC A61F 2/08; A61F 2210/0004; A61F 2210/0014; A61F 2230/0069

(57) ABSTRACT

A device is provided for supporting tissue. The device includes a body operable to couple with the tissue. The body forms a lumen operable to receive at least a portion of the tissue. The body forms a plurality of apertures to form a matrix so that (1) when the body transitions towards a contracted configuration, a diameter of the lumen increases, and (2) when the body transitions towards an elongated configuration, the diameter of the lumen decreases.

### CROSS-REFERENCE TO RELATED APPLICATIONS

This application claims priority benefit of U.S. Provisional Application No. 63/640,928 filed May 1, 2024, which is incorporated herein by reference in its entirety for all purposes.

### FIELD

The present disclosure relates generally to a device for tissue support. In at least one example, the present disclosure relates to a device that is operable to provide support and/or reinforcement to tissue such as soft tissue and/or connective tissue.

### BACKGROUND

Tissue, such as ligaments, tendons, and nerves, stretch during movement as they adapt to the forces and tensions placed on them, allowing for flexibility and mobility while maintaining structural integrity. For example, tendons and ligaments, which are responsible for connecting muscles to bones, stabilizing joints, and producing motion with muscle contraction, often experience mechanical stress and strain that can result in injury and impair their healing when damaged. Similarly, nerve injuries require careful repair to restore function and avoid complications such as loss of sensation or motor control.

### BRIEF DESCRIPTION OF THE DRAWINGS

Implementations of the present technology will now be described, by way of example only, with reference to the attached figures.

FIG. 1A illustrates a diagram of a device providing support for a tendon.

FIG. 1B illustrates a diagram of the device providing support for a nerve.

FIG. 2A illustrates a side view of an example of the device.

FIG. 2B illustrates a top view of the device of FIG. 2A.

FIG. 3 illustrates an example of the device.

FIG. 4A illustrates the device in a relaxed configuration.

FIG. 4B illustrates the device in a contracted configuration.

FIG. 4C illustrates the device in an elongated configuration.

FIG. 5 is a flow chart of a method of coupling the device with tissue.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the examples described herein. However, it will be understood by those of ordinary skill in the art that the examples described herein can be practiced without these specific details. In other instances, methods, procedures and components have not been described in detail so as not to obscure the related relevant feature being described. Also, the description is not to be considered as limiting the scope of the examples described herein. The drawings are not necessarily to scale and the proportions of certain parts may be exaggerated to better illustrate details and features of the present disclosure.

The device is operable to couple with tissue (e.g., tendons, ligaments, and/or nerves) that needs support and/or repair and offers additional strength and stability to the tissues, promoting better alignment, cell growth, and tissue integration. By improving the healing environment, the device can assist in restoring the function and durability of the tissues, minimizing the risk of reinjury and enhancing long-term recovery outcomes.

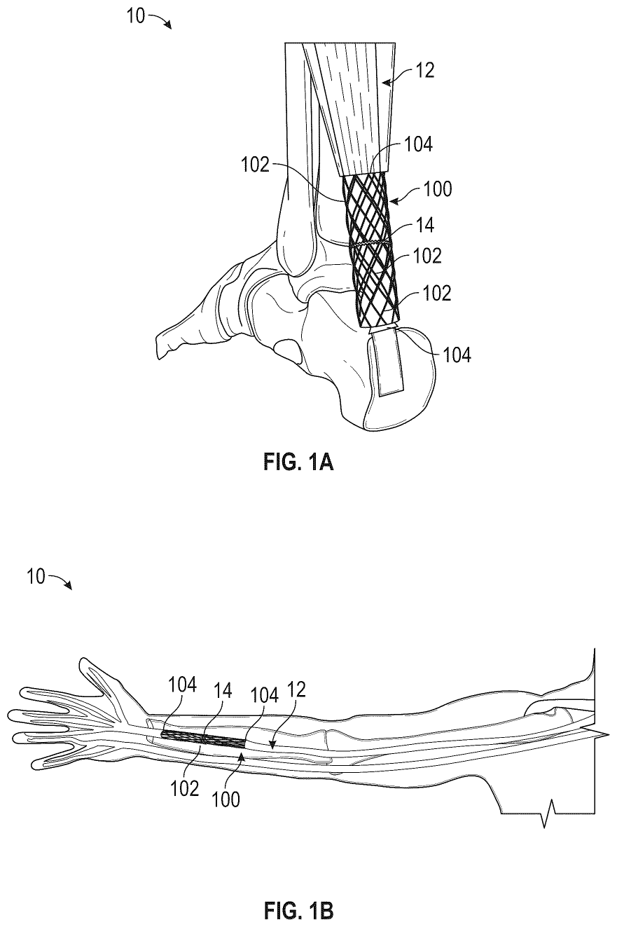

Referring now to FIG. 1A, the device 100 can be used for supporting tissue 12 such as ligaments and/or tendons. For example, the tissue 12 may have ruptured and/or been injured, and further support is needed to the repair site 14. For example, the repair site 14 can be the location where the tissue 12 has been coupled together after a rupture and/or tear. In some examples, the repair site 14 can be where the tissue 12 has been weakened and, in some examples, can be under threat of rupture and/or tear.

Surgical repair techniques for injuries to tissue 12 such as tendon and ligament are focused on re-establishing alignment by suturing the ruptured ends of the tissue 12 together. These suture reconstructions must be performed using very specific and intricate suture techniques to help withstand tension and load and prevent gapping and rupture. In some cases, when there is a significant gap or defect, tendon/fascia/tissue grafts can be harvested from the patient and used as an interposition graft to bridge the defect. In some instances, particularly for tendons 12, as illustrated in FIG. 1A, surgeons can also use nearby tendons from expendable muscles as a transfer to provide some function and/or motion. However, these techniques often require even greater complexity with respect to suturing, including weaving the tendons and/or tissue 12 within one another to add strength, which can create bulky tissue scar 14 that could limit motion and gliding due to adhesions. In addition, although tendon transfer can restore function, these transfers often result in a mismatch between strength, excursion and tensile properties of the native tissue, and result in additional donor site morbidity.

The goals of tendon and ligament repair are to promote healing and minimize scarring to allow for strength, stability, range of motion and motion of a joint. Tendon/Ligament healing can take 4-12 months to complete, which is substantial, and can affect quality of life, functional recovery, and lead to complications and/or long-term functional disability. Despite advances in the materials and methods to treat these injuries, complications are common. The most common complication is adhesion formation, which limits active range of motion. Other complications include joint contracture, tendon rupture, and disrupted mechanical forces resulting in suboptimal joint function. Gap formation from tension is also a common complication after tendon repair and is associated with adhesion formation, tendon rupture, and decreased strength. Rupture typically occurs soon after the repair when the tendon is the weakest. Meta-analyses report that these complications occur frequently and can affect up to 1 out of every 20 cases.

Referring to FIG. 1B, the device 100 can be utilized to assist with healing, reinforcement, and/or support for tissue 12 such as nerves. While FIGS. 1A and 1B illustrate the device 100 being utilized with tissue 12 that includes nerves, tendons, and ligaments, other connective tissue and soft tissue 12 can be supported without deviating from the scope of the disclosure.

Central and peripheral nerves 12 are critical signaling structures in the human body that provide us with the electrical impulse to move, feel, and function. A nerve injury (e.g., repair site 14) can affect this function and lead to devastating physical, functional, and sensory deficits. Although nerve injuries 14 can be caused in several ways, they are most commonly caused by trauma, medical conditions, and/or autoimmune diseases. Traumatic nerve injuries 14 include those occurring as a result of accidents, falls, sports injuries, compression, crush injury, and penetrating injuries. Damaged nerves 12 result in symptoms that depend upon they type of nerve 12 injured (e.g. sensory, motor, or autonomic nerves), and can include muscle paralysis or weakness, numbness, pain, or sensory changes, or over/under-activity of subconscious functions like sweating, blood pressure, even gastrointestinal symptoms. When a nerve 12 is damaged, the nerve 12 should be repaired where possible, particularly when it relates to peripheral motor nerves that are cut or crushed.

The most common repair techniques for crushed or transected nerves 12 include suture repair and/or use of tubes/conduits when a gap or nerve deficit exists (e.g., repair site 14). Depending upon the timing of the injury, the type of nerve 12, and the length of the gap, the outcomes can vary significantly. A successful nerve repair requires healthy proximal and distal nerve stumps, proper alignment of these stumps, management of any gaps, and a connection/coaptation, that is both tension-free and atraumatic. Excessive tension on the nerve repair site 14 can disrupt blood supply, affect the axonal growth, and ultimately the outcome. Likewise, large gaps (for example, greater than 2-3 centimeters) are often quite challenging because of the distance with which the axons must grow to overcome the gap. In many of these cases, surgeons elect to utilize autologous donor nerves, or allogenic cadaver nerve grafts, to overcome the gap 14. Although these grafts may result in some return of nerve function, they are associated with donor site morbidity, potential immune response and rejection, and incomplete recovery.

Conventional nerve tubes have been used to assist with limitations related to nerve gaps and disadvantages of autologous nerve grafting. Although these tubes have been successfully used, they still have limitations including issues related to efficacy dependent upon gap length, nerve diameter mismatch, and the risk of encapsulation or scar tissue formation. Likewise, conventional nerve tubes often need to be fixated to the perineurium to prevent migration, which can be traumatic.

Vein grafts have been used as nerve conduits with some success. Reports indicate these grafts may work as well as autogenous nerve grafts for short gaps, in small nerves. However, they are also associated with donor site morbidity, and limited efficacy in large gaps.

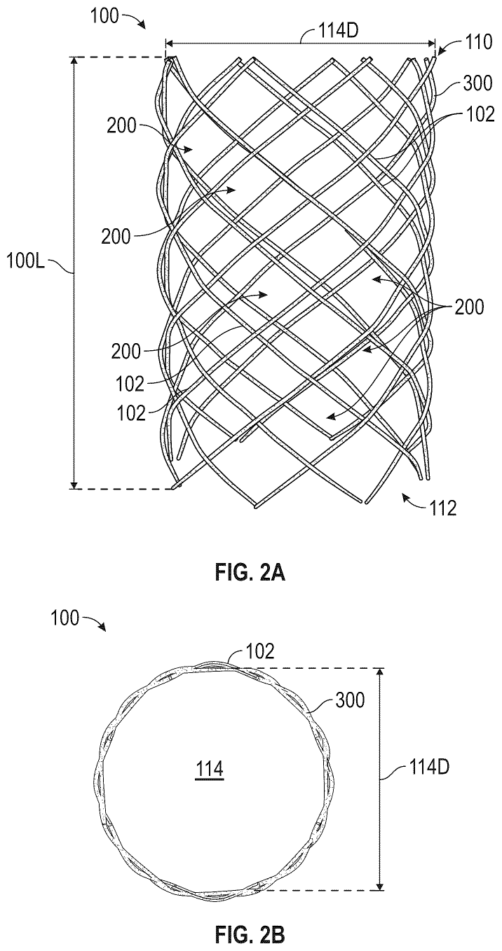

The device 100 for supporting the tissue 12: (1) is biocompatible and potentially biodegradable; (2) supports cell attachment and growth; (3) has high surface area; (4) promotes healing; (5) reduces inflammation; (6) has strength that mimics native tissue properties. As shown in FIGS. 1A and 1B, the device 100 can have a first end 104 and a second end 104 opposite the first end 104 that are coupled with a first section and a second section of the tissue 12. For example, the device 100 can be operable to couple with the tissue 12 via coupling mechanisms, including but not limited to, compression, elastic recoil of the device 100 (e.g., the body 110 as illustrated in FIGS. 2A-4C) to a resting configuration, adhesive, suture, clip, hook, barb, staple, tack, screw, pin, frictional elements, piercing features, cerclage band, purposeful elongation of the device 100 (e.g., the body 110 as illustrated in FIGS. 2A-4C) via tension to compress and grip the tissue 12. Other suitable mechanisms to couple the device 100 with the tissue 12 can be utilized without deviation from the scope of the disclosure. The first section and second section can be opposing sides of the tissue 12 in relation to the repair site 14. Accordingly, the device 100 can serve as additional support when the tissue 12 extends and is under stress, reducing the stress on the repair site 14. Therefore, the repair site 14 of the tissue 12 can more effectively and efficiently heal.

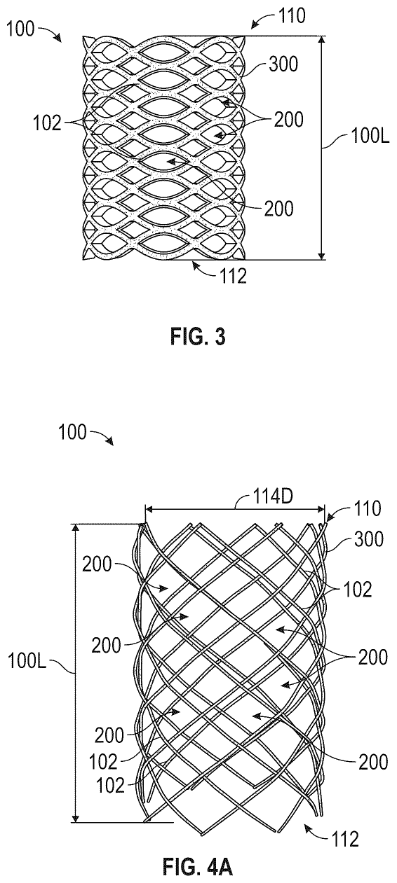

As shown in FIGS. 2A, 2B, and 3, the device 100 can include a body 110 that is operable to couple with the tissue 12. The body 110 can form a lumen 114 operable to receive at least a portion of the tissue 12. For example, the lumen 114 can be operable to receive the first section and second section of the tissue 12 as well as the repair site 14. In some examples, the body 110 can have substantially a tubular shape so that the body 110 can substantially match the shape of the tissue 12 received therein. While the body 110 as illustrated herein has a tubular shape, the body 110 can have any suitable geometry to substantially match the tissue 12 received in the lumen 114. Accordingly, the body 110 can provide high surface area and contact to allow for even and distributed forces to be dispersed therethrough. In some examples, the body 110 can be in the form of a sheet that can then be wrapped and/or rolled to surround, encircle, and/or envelope the tissue 12 such as tendon, nerve, ligament, muscle, and/or bone. The body 110 can allow for connective tissue 12 and/or soft-tissue 12 like tendon, nerve, ligament, muscle, bone, vessels, graft materials, and/or intestines, to pass through the lumen 114. Accordingly, the length 100L of the body 110, the thickness, and/or the width 114D (and/or diameter 114D) of the body 110 can be manufactured depending on upon the tissue 12 to be supported.

In at least one example, the body 114 can be at least partially made of biocompatible material for implant in a patient's body. In some examples, the biocompatible material can include one or more of the following: permanent material, metals, biomaterial, biopolymer, polymer, bioresorbable material, absorbable material, synthetic material, biologic material, and/or any combination thereof. For example, the biocompatible material could include, but not be limited to, one or more of the following: collagen, xenograft tissue, porcine small intestine submucosa (SIS), acellular dermis, silk, hyaluronic acid, elastin, chitosan, P4HB, citrate-based elastomers, polydioxanone (PDS), PLA, PGA, PLLA, PCL, polypropylene, ePTFE, or polyglactin, stainless steel, titanium, nitinol, and/or any combination thereof.

In some examples, at least a portion of the body 110 can include at least one supplemental component 300. The supplemental component 300 can include cells, medicaments, drugs, growth factors, hormones, electrical stimulators, electrodes, biosensors, additives for healing support, additives for antimicrobial effects, and/or additives for anti-inflammatory effects. In some examples, the supplemental component 300 can be dipped, coated, and/or loaded onto and/or into the body 110.

As shown in FIGS. 2A-4C, the body 110 can form a plurality of apertures 200 to form a matrix 102. In some examples, as illustrated in FIGS. 2A and 2B, the matrix 102 can include a plurality of fibers. In some examples, as illustrated in FIG. 3, the matrix 102 can be formed by an integral structure. The matrix 102 can be formed, for example, by manufacturing mechanisms such as braiding, weaving, knitting, 3-D printing, and/or assembly necessary to create the mechanical strength and properties necessary.

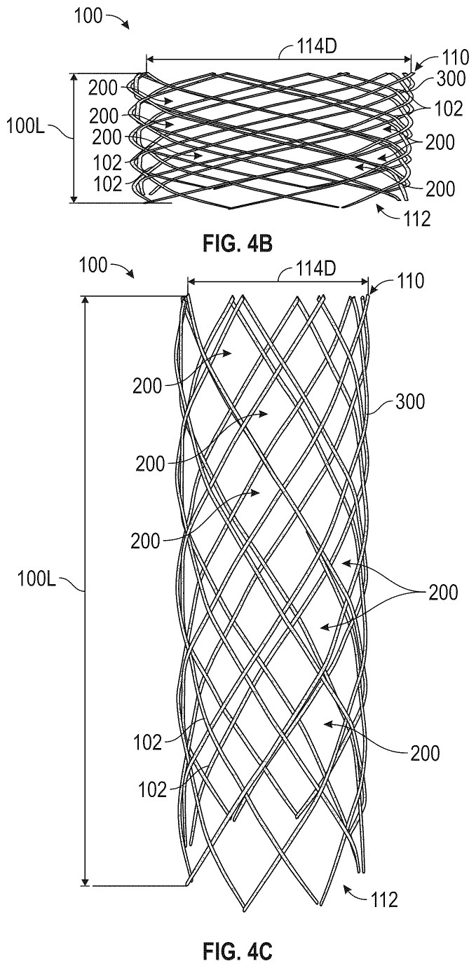

Referring to FIGS. 4A-4C, the matrix 102 of the body 110 can be formed so that (1) when the body 110 transitions towards a contracted configuration, a diameter 114D of the lumen 114 increases, and (2) when the body 110 transitions towards an elongated configuration, the diameter 114D of the lumen 114 decreases. FIG. 4A illustrates that the matrix 102 of the body 110 is in a relaxed configuration under no tensile stress. When the body 110 transitions towards the contracted configuration (as shown in FIG. 4B), the length 110L of the body 110 decreases. When the body 110 transitions towards the elongated configuration (as shown in FIG. 4C), the length 110L of the body 110 increases.

With such a configuration, the matrix 102 can be operable to disperses tensile and/or traction forces received by the tissue 12 into radial compression forces thereby narrowing of the lumen 114 of the body 110. This effect allows for matching within a predetermined threshold of the diameter 114D of the body 110 to the diameter of the interposed tissue 12 based upon length. Accordingly, the body 110 and the matrix 102 allows for minor adjustment of the size of the lumen 114 through longitudinal stretch and/or elongation such that the body 110 construct can conform to the shape and size of the tissue 12 within the lumen 114 and grip, or come into firm contact, with the tissue 12. This can enable the user to effectively set the appropriate tension load within the device 110 to protect the interposed tissue 12 from unexpected/accidental tension and/or motion.

Once the body 110 is appropriately positioned and elongated to create firm grip and/or contact with the tissue 12 received in the lumen 114, the body 110 can then be fixed proximally and distally to the tissue 12 in this position through a plurality of mechanisms, including but not limited to adhesive, suture, clip, hook, barb, staple, tack, frictional elements within the polymer mesh, piercing features, cerclage band, etc.

With the body 110 is fixed to the tissue 12, the device 100 has the mechanical properties necessary to provide reinforcement and strength to the tissue 12 through rest, motion, and/or tension, to support healing and prevent dehiscence, gapping, rupture, unwanted motion, separation, or excessive scarring. Through action of the device 100, elongation and/or traction disperses the motion and force received by the tissue 12 into radial compressive forces to narrow the inner diameter 114D and prevent motion and/or displacement of the underlying tissue 12. Accordingly, when deployed, the device 100 is elongated to a desired length resulting in the lumen 114 narrowing to an acceptable diameter 114D that mimics and/or matches the external diameter and/or width of the intervening tissue 12 being supported. For example, when the tissue 12 receives tensile forces such as stretching or contracting, the body 110 stretches or contracts along with the tissue 12, and the diameter 114D of the lumen 114 correspondingly contracts (when the length 110L increases) and/or expands (when the length 110L decreases). This action resists excessive traction/elongation/tension on the underlying tissue 12 to reduce the risk of rupture, gapping, or tension on the repair site 14. For example, when the tissue 12 stretches, the repair site 14 is under additional stress. The body 110 of the device 100 then stretches as well, and with the reduction of the diameter 114D, the device 100 actually increases the grip or coupling with the tissue 12 to prevent excessive stretch or tension on the repair site 14. Additionally, the external aspect of the device 100 protects the underlying repair site 14 from scarring and adhesions.

In some examples, the body 110 can be disposed within, at least partially received in, and/or coupled with one or more layers. For example, the body 110 can at least partially coated and/or covered in an outer layer and/or an inner layer (or sandwiched between both the outer layer and the inner layer). In some examples, the outer layer and/or the inner layer can provide a smooth surface. In some examples, the outer layer and/or the inner layer can provide an adhesion barrier. In some examples, the outer layer and/or the inner layer can be coated with medicaments and/or other suitable additives.

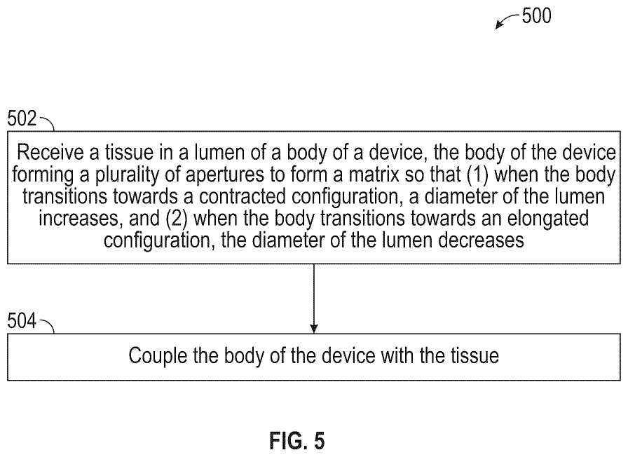

Referring to FIG. 5, a flowchart is presented in accordance with an example embodiment. The method 500 is provided by way of example, as there are a variety of ways to carry out the method. The method 500 described below can be carried out using the configurations illustrated in FIGS. 1A-4C, for example, and various elements of these figures are referenced in explaining example method 500. Each block shown in FIG. 5 represents one or more processes, methods, or subroutines, carried out in the example method 1000. Furthermore, the illustrated order of blocks is illustrative only and the order of the blocks can change according to the present disclosure. Additional blocks may be added or fewer blocks may be utilized, without departing from this disclosure. The example method 500 can begin at block 502.

At block 502, a tissue is received in a lumen of a body of a device. The body of the device can form a plurality of apertures to form a matrix so that (1) when the body transitions towards a contracted configuration, a diameter of the lumen increases, and (2) when the body transitions towards an elongated configuration, the diameter of the lumen decreases. When the body transitions towards the contracted configuration, the length of the body decreases. When the body transitions towards the elongated configuration, the length of the body increases.

In at least one example, the body is at least partially made of a biocompatible material for implant in a patient's body.

At block 504, the body of the device is coupled with the tissue. The body can have a first end and a second end opposite the first end. The first end can be coupled with a first section of the tissue, and the second end can be operable to couple with a second section of the tissue.

In at least one example, the body can be coupled with the tissue via one or more of the following: compression, elastic recoil of the body to a resting configuration, adhesive, suture, clip, hook, barb, staple, tack, screw, pin, frictional elements, piercing features, cerclage band, purposeful elongation of the body via tension to compress and grip the tissue.

Once the device is coupled with the tissue, the matrix can be operable to disperse tensile and/or traction forces received by the tissue into radial compression forces

The disclosures shown and described above are only examples. Even though numerous properties and advantages of the present technology have been set forth in the foregoing description, together with details of the structure and function of the present disclosure, the disclosure is illustrative only, and changes may be made in the detail, especially in matters of shape, size and arrangement of the parts within the principles of the present disclosure to the full extent indicated by the broad general meaning of the terms used in the attached claims. It will therefore be appreciated that the examples described above may be modified within the scope of the appended claims.

1. A device for supporting tissue, the device comprising:
a body having a first end and a second end, the body operable to receive a first portion and a second portion of a tissue, wherein the first portion and the second portion of the tissue are on opposing sides of a repair site of the tissue,
wherein the body forms a lumen operable to receive the first portion and the second portion of the tissue so that the repair site is received in the lumen,
wherein the first end of the body is operable to be coupled with the first portion of the tissue, and the second end of the body is operable to be coupled with the second portion of the tissue,
wherein the body forms a plurality of apertures to form a matrix so that (1) when the body transitions towards a contracted configuration, a diameter of the lumen increases, and (2) when the body transitions towards an elongated configuration, the diameter of the lumen decreases, to reduce stress on the repair site.

2. The device of claim 1, wherein when the body transitions towards the contracted configuration, a length of the body decreases.

3. The device of claim 1, wherein when the body transitions towards the elongated configuration, a length of the body increases.

4. (canceled)

5. The device of claim 1, wherein the body is operable to couple with the tissue via one or more of the following: compression, elastic recoil of the body to a resting configuration, adhesive, suture, clip, hook, barb, staple, tack, screw, pin, frictional elements, piercing features, cerclage band, purposeful elongation of the body via tension to compress and grip the tissue.

6. The device of claim 1, wherein the body has substantially a tubular shape.

7. The device of claim 1, wherein the body is at least partially made of a biocompatible material for implant in a patient's body.

8. The device of claim 7, wherein the biocompatible material includes one or more of the following: permanent material, metals, biomaterial, biopolymer, polymer, bioresorbable material, absorbable material, synthetic material, biologic material, and/or any combination thereof.

9. The device of claim 7, wherein the biocompatible material includes one or more of the following: collagen, xenograft tissue, porcine small intestine submucosa (SIS), acellular dermis, silk, hyaluronic acid, elastin, chitosan, P4HB, citrate-based elastomers, polydioxanone (PDS), PLA, PGA, PLLA, PCL, PLGA, polyethylene, polylactide, polypropylene, ePTFE, polyurethane, polyglactin, stainless steel, titanium, nitinol, and/or any combination thereof.

10. The device of claim 1, wherein at least a portion of the body includes at least one supplemental component which includes cells, medicaments, drugs, growth factors, hormones, electrical stimulators, electrodes, biosensors, additives for healing support, additives for antimicrobial effects, and/or additives for anti-inflammatory effects.

11. The device of claim 10, wherein the at least one supplemental component is dipped, coated, and/or loaded onto and/or into the body.

12. The device of claim 1, wherein the matrix is operable to disperses tensile and/or traction forces received by the tissue into radial compression forces.

13. The device of claim 1, wherein the matrix is formed by at least one of the following: braiding, weaving, knitting, 3-D printing, and/or assembly.

14. A method comprising:
receiving a tissue in a lumen of a body of a device so that a repair site is received in the lumen of the body, wherein a first portion and a second portion of the tissue are on opposing sides of the repair site of the tissue;
coupling a first end of the body of the device with the first portion of the tissue;
coupling a second end of the body of the device with the second portion of the tissue,
wherein the body of the device forms a plurality of apertures to form a matrix so that (1) when the body transitions towards a contracted configuration, a diameter of the lumen increases, and (2) when the body transitions towards an elongated configuration, the diameter of the lumen decreases, to reduce stress on the repair site.

15. The method of claim 14, further comprising:
dispersing, by the matrix, tensile and/or traction forces received by the tissue into radial compression forces.

16. The method of claim 14, wherein when the body transitions towards the contracted configuration, a length of the body decreases.

17. The method of claim 14, wherein when the body transitions towards the elongated configuration, a length of the body increases.

18. (canceled)

19. The method of claim 14, wherein the body is operable to be coupled with the tissue via one or more of the following: compression, elastic recoil of the body to a resting configuration, adhesive, suture, clip, hook, barb, staple, tack, screw, pin, frictional elements, piercing features, cerclage band, purposeful elongation of the body via tension to compress and grip the tissue.

20. The method of claim 14, wherein the body is at least partially made of a biocompatible material for implant in a patient's body.
